(19) United States
(12) Patent Application Publication
(10) Pub. No.: US 2025/0367454 A1
(43) Pub. Date: Dec. 4, 2025

(54) MODULE FOR CALCULATING A CARDIAC RHYTHM SETPOINT FOR AN IMPLANTABLE PACEMAKER CONTROLLED DEPENDING ON THE PATIENT'S ACTIVITY

(71) Applicant: CAIRDAC, Antony (FR)

(72) Inventor: Alaa MAKDISSI, Paris (FR)

(21) Appl. No.: 19/199,604

(22) Filed: May 6, 2025

(30) Foreign Application Priority Data
May 31, 2024 (EP) 24315256

(51) Int. Cl. A61N 1/365

(52) U.S. Cl. CPC A61N 1/36585

(57) ABSTRACT

The module comprises: a conversion stage receiving as an input a sampled activity signal representative of the patient's instantaneous activity and outputting a first target value by application of a predetermined activity/cardiac rhythm function; a low-pass recursive digital filter calculating over a predetermined duration a moving average of the first target value issued by the conversion stage and outputting a second target value; and a combiner stage receiving as an input the first target value and the second target HR value issued by the first low-pass digital filtering stage, determining the maximum of both target values and outputting the setpoint value to control the pacing frequency depending on the patient's activity.

### BACKGROUND OF THE INVENTION

### Technical field

The invention relates to implanted medical devices, in particular pacemakers that continuously monitor the patient's rhythm and issues as needed to the heart electrical pacing pulses to alleviate a myocardial sinus rhythm disorder.

The invention is particularly advantageously applicable-but not limited-to autonomous implantable devices of the “leadless capsule” type, which are implantable devices having no physical connection (lead) with a remote device.

The capsule comprises various electronic circuits, sensors, etc., as well as wireless communication transmission/reception means for the remote exchange of data, the whole being integrated in a very small size body able to be implanted at sites of difficult access or leaving little available space, such as the ventricle apex or the inner wall of the atrium.

One of the critical aspects of these miniaturized devices is the power autonomy, and consequently the consumption of the electronic circuits, which must be as low as possible.

With a leadless implant, taking into account the very small dimensions, it is not possible to use a conventional battery, even a high-density one. Hence, a self-powering system is provided, with an energy harvester that collects the mechanical energy resulting from various movements undergone by the implant body at the rhythm of heartbeats, and converts this mechanical energy into electrical energy by means of a suitable transducer, to charge an integrated battery and to power the various circuits and sensors of the device. This power supply system allows the device to operate in full power autonomy for its whole lifetime, of about 8 to 10 years.

WO 2019/001829 A1 (Cairdac) describes an example of such an autonomous leadless intracardial capsule provided with an integrated energy harvester.

The invention is nevertheless not limited to this particular type of implant; it can also be applied to other types of pacemakers powered by a battery, the life of which must be preserved by minimizing the overall power consumption of the device.

Pacing is generally carried out in VVIR or “rate-responsive” mode, which means that if the rhythm is paced rather than spontaneous (sinusal), the rate of the pacing pulses must be modulated according to the patient's physical activity, with a lower rate when the patient is inactive and a higher rate for increased physical activity. The rate of the so-controlled paced rhythm will vary between a minimum rate called “baseline rate” (HRₘᵢₙ) and a maximum rate (HRₘₐₓ) specific to the patient, which defines a top limit for the pacing rate calculated by the control algorithms.

The pacing rate control must also be as physiological as possible, replicating the sinus rhythm of a healthy heart with (i) a rapid increase in rhythm in the event of a sudden increase in activity (the patient getting up and walking, climbing stairs, etc.), and (ii) then a return to the base rate that is on the contrary very gradual and slow, of the order of a few tens of seconds or several minutes after detection of lesser activity (the patient who has finished climbing the stairs but still needs to recover from the effort, etc.). The variation rate of the controlled pacing rate, i.e. the shape (linear or not) of the rate/effort characteristic, can also be adapted to the patient's physical activity.

To carry out this control function, the level of the patient's instantaneous physical activity is typically measured by a so-called “activity sensor” or “G sensor” which is typically an accelerometer, most often a 3D accelerometer.

This type of sensor, that issues an accelerometric signal with very rapid variations of very large amplitude, is to be distinguished from so-called “physiological sensors” or “effort sensors” such as the minute-ventilation sensors or “MV sensors” which issue a slowly varying signal representative of the patient's metabolic needs, and that are applicable to the case of an autonomous leadless capsule (they are based on the measurement of a transthoracic impedance between the tip of an intracardiac lead and a remotely located case of a pacemaker generator).

### State of the Art

The problem of the invention is that of the continuous (cycle-by-cycle) calculation of the optimum rate for the pacing pulses to be issued by the device, this rate having ideally to be calculated for each cardiac cycle in order to optimize the adaptation to the patient's activity. US 2020/0147396 A1 (Shelton et al./Medtronic) describes a leadless pacemaker provided with an accelerometric sensor detecting the patient's movements and level of activity. The accelerometric sensor signal is suitably sampled and filtered, then processed so as to determine on each new cardiac cycle a target rate for the device control.

To reduce the consumption of the integrated microprocessor that performs these operations, the latter is made active only for a sampling period of limited duration, after expiry of a blanking period during which sampling is suspended.

However, this solution is not optimum. Namely:
at each cardiac cycle, the processor wake-up from sleep mode to active mode is not immediate: it requires several tens of processor cycles, i.e. from a few microseconds to a few milliseconds, before being able to begin recalculating the pacing rate;
during this wake-up phase, the consumption of the processor is at least equivalent to its consumption during the active phase, even though it is not yet capable of sampling and processing the signal;
the algorithm is based on a cumulative total of 1000 previous values of the pacing rate (i.e. a duration of about 15 minutes) to take into account hysteresis, i.e. the fact that the variation law is not the same in a situation of patient's activity increase (requiring a rapid acceleration of the pacing rate) and in the case of decrease of this activity (requiring a very progressive and slow return to the base rate). This retrospective analysis over a long duration requires performing about 2000 processor cycles before being able to stabilize the control, causing an almost doubling of the power consumption of a controlled algorithm (VVIR) compared with a simple, non-controlled algorithm (VVI);
finally, the presence of a blanking period results in that part of the accelerometric signal remains masked and will never be taken into account for calculating the control, which reduces accordingly the accuracy of the latter.

The aim of the invention is to overcome these drawbacks and limitations by proposing a technique for continuously adapting the cardiac rhythm, advantageously applicable to a leadless implant, requiring only very low consumption by the device's internal circuits, without thereby degrading the control performance compared with known techniques.

### SUMMARY OF THE INVENTION

To solve the different problems and achieve the above-mentioned aims, the invention essentially proposes a module for calculating a cardiac rhythm value comprising, in a manner known per se, a module for calculating a cardiac rhythm, HR, or cardiac interval, RR, setpoint value, intended to control depending on the patient's activity the rate of the pacing pulses issued by an active implantable medical device, wherein the HR or RR setpoint value is continuously determined by digital processing of a sampled activity signal representative of the patient's instantaneous activity. Characteristically of the invention, this module further comprises:
a conversion stage, receiving as an input a current value of the sampled activity signal and outputting a first target HR or RR value, obtained by application of a predetermined activity vs. cardiac rhythm function, or respectively activity vs. cardiac interval function, to a current value of the sampled activity signal;
a first low-pass digital filtering stage, comprising a recursive filter capable of calculating, over a first predetermined duration, a first moving average of the first target HR or RR value issued by the conversion stage, outputting a second target HR or RR value; and
a combiner stage, receiving as an input (i) the first target HR or RR value issued by the conversion stage, and (ii) the second target HR or RR value issued by the first low-pass digital filtering stage, and adapted to determine the maximum of the two target HR values, or respectively the minimum of the two target RR values, received as an input, outputting the HR or RR setpoint value, to control the pacing rate depending on the patient's activity.

According to various advantageous subsidiary features:
the module is integrated to an ASIC circuit;
the module comprises a microcontroller operating without being put to sleep between two consecutive cardiac cycles;
the module further comprises a second low-pass digital filtering stage, comprising a recursive filter capable of calculating, over a second predetermined duration shorter than the first predetermined duration, a second moving average of the first target HR or RR value, issued by the conversion stage, so as to eliminate parasitic high-frequency noise components that are not representative of the patient's instantaneous activity, the combiner stage and the first low-pass digital filtering stage receiving as an input the first target HR or RR value, after the latter has been filtered by the second low-pass digital filtering stage;
the predetermined activity vs. cardiac rhythm function, or respectively the activity vs. cardiac interval function, is a linear function;
to determine the patient's recovery time after a period of intense activity, the first predetermined duration for the first low-pass digital filtering stage to calculate the first moving average is between 120 and 600 seconds;
in this latter case, to determine the reactivity of the cardiac rhythm or cardiac interval adaptation while eliminating the activity noises of very short duration, the second predetermined duration for the second low-pass digital filtering stage to calculate the first moving average is between 1 and 2 seconds;
the recursive filter of the first and/or the second low-pass digital filtering stage is an exponential recursive filter of the 1st order; and/or
the recursive filter of the first and/or the second low-pass digital filtering stage is a filter capable of calculating a moving average without division nor multiplication, in particular a filter capable of operating by shifting bits of the digital representation of the target HR or RR value at the input of the filtering stage.

The invention also relates to an active medical device of the autonomous implantable capsule type which houses, in a device body, an electronic unit comprising:
a pacing circuit, capable of providing pacing pulses;
a circuit capable of issuing a signal representative of the instantaneous activity of a patient wearing the device;
a module for sampling or extracting a sampled activity signal from the signal representative of the patient's instantaneous activity; and
a module for calculating a HR or RR setpoint value as mentioned hereinabove, to control depending on the patient's activity the rate of the pacing pulses issued by the pacing circuit.

### BRIEF DESCRIPTION OF THE DRAWINGS

An exemplary embodiment of the present invention will now be described with reference to the appended drawings, in which the same references denote identical or functionally similar elements throughout the figures.

FIG. 1 illustrates a medical device of the leadless capsule type in its environment, implanted in the apex of the right ventricle of a patient.

FIG. 2 schematically shows the main functional blocks constituting the leadless capsule.

FIGS. 3a and 3b illustrate the way to control as physiologically as possible the patient's activity cardiac rhythm, in a manner comparable to the natural adaptation in a healthy patient.

FIG. 4 is a schematic representation, in block form, of the various digital signal processing stages of a module according to the invention for issuing, from a sampled activity signal from an accelerometer, a cardiac rhythm setpoint value to be applied to the input of a control stage of the pacing circuit of an implanted device.

FIGS. 5a to 5d are chronograms illustrating the sampled signal values recorded at different levels of the module according to the invention illustrated in FIG. 4, during detected variations of the patient's activity.

FIG. 6 is a cardiac rhythm vs. activity level characteristic, corresponding to the activity variations illustrated in FIGS. 5a to 5d.

### DETAILED DESCRIPTION OF PREFERENTIAL EMBODIMENTS OF THE INVENTION

An exemplary embodiment of the device of the invention will now be described, in an application to an autonomous implantable capsule intended to be implanted into a heart cavity.

As indicated hereinabove, this particular application is given only as an example of embodiment and does not limit the invention, the teachings of which can be applied to many other types of autonomous devices incorporating or not an energy harvester of the PEH type.

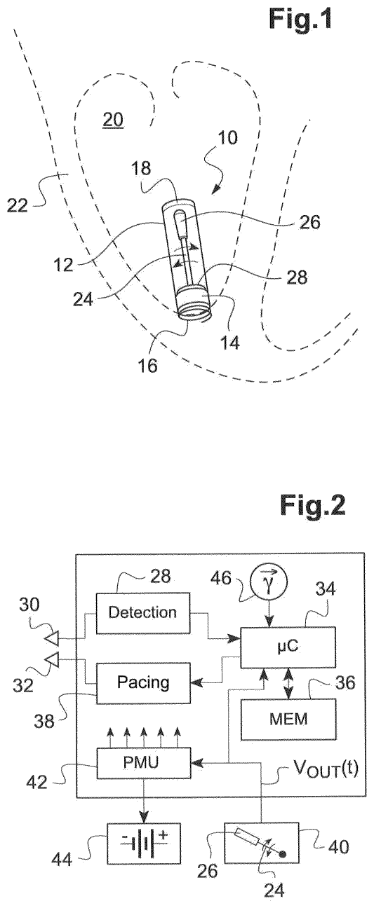

FIG. 1 shows a leadless capsule device 10 in an application to cardiac pacing.

Capsule 10 has the external form of an implant with a cylindrical, elongated tubular body 12 enclosing the various electronic and power supply circuits of the capsule, as well as an energy harvester with a pendular unit. The typical size of such a capsule is about 6 mm diameter for about 25 to 40 mm length.

Tubular body 12 has, at its front (distal) end 14, a protruding anchoring element, for example a helical screw 16, to hold the capsule on the implantation site. Other anchoring systems can be used, and do not change in any way the implementation of the present invention. The opposite (proximal) end 18 of capsule 10 is a free end, which is only provided with means (not shown) for the temporary connection to a guide-catheter or another implantation accessory used for implantation or explantation of the capsule, which is then detached from the latter.

In the example illustrated in FIG. 1, leadless capsule 10 is an endocavitary implant implanted into a cavity 20 of myocardium 22, for example at the apex of the right ventricle. As an alternative, still in an application to cardiac pacing, the capsule can also be implanted on the interventricular septum or on an atrial wall, or also be an epicardial capsule placed on an external region of the myocardium, these different implantation modes not changing in any way the implementation of the present invention. To perform the detection/pacing functions, an electrode (not shown) in contact with the heart tissue at the implantation site collects the heart depolarization potentials and/or applies pacing pulses. In certain embodiments, the function of this electrode can be provided by anchoring screw 16, which is then an active screw, electrically conductive and connected to the detection/pacing circuit of the capsule.

Leadless capsule 10 is further provided with an energy harvesting module, so-called “PEH”, comprising an inertial pendular unit that oscillates, inside the capsule, following the various external stresses to which the capsule is subjected. These stresses may result in particular from: movements of the wall to which the capsule is anchored, which are transmitted to tubular body 12 by anchoring screw 16; and/or blood flow rate variations in the environment surrounding the capsule, which produce oscillations of tubular body 12 at the rhythm of the heartbeats; and/or various vibrations transmitted by the heart tissues.

The pendular unit consists in a piezoelectric beam 24 clamped at one of its ends and whose opposite, free end is coupled to a mobile inertial mass 26. Piezoelectric beam 24 is an elastically deformable flexible beam that constitutes, with inertial mass 26, a pendular system of the mass-spring type. Due to its inertia, mass 26 subjects beam 24 to a deformation of the vibratory type on either side of a neutral or non-deformed position corresponding to a stable rest position in the absence of any stress.

FIG. 2 is a synoptic view of the various electric and electronic circuits integrated to the leadless capsule, presented as functional blocks. Block 28 denotes a heart depolarization wave detection circuit, which is connected to a cathode electrode 30 in contact with the heart tissue and to an associated anode electrode 32, for example a ring electrode formed on the tubular body of the capsule. Detection block 28 comprises filters and means for analog and/or digital processing of the collected signal. The thus processed signal is applied to the input of a microcomputer 34 associated with a memory 36. The electronic unit also includes a pacing circuit 38 operating under the control of microcomputer 34 to issue, as needed, to the electrode system 30, 32 myocardial pacing pulses.

Further, an energy harvesting circuit or PEH 40 is provided, comprising the pendular unit formed by piezoelectric beam 24 and inertial mass 26, described hereinabove with reference to FIG. 1.

Piezoelectric beam 24, which ensures a mechanical-electrical transducer function, converts into electrical charges the mechanical stresses undergone and produces a variable electrical signal V_{OUT}(t), which is an alternating signal oscillating at the natural oscillation rate of the pendular beam 24/mass 30 unit, and at the rhythm of the successive beats of the myocardium to which the capsule is coupled. This variable electrical signal V_{OUT}(t) is issued to a power management circuit or PMU 42, which rectifies and regulates the signal V_{OUT}(t) so as to output a stabilized direct voltage or current for powering the various electronic circuits and charging an integrated battery 44.

The leadless capsule also integrates a cardiac activity sensor 46 such as a 1D, or preferably 3D, accelerometer of the piezoelectric, piezoresistive or capacitive type, such as MEMS.

Sensor 46 continuously provides a composite signal containing (i) components representative of the instantaneous activity of the patient wearing the device and (ii) components representative of the acceleration, due to heartbeats, of the wall on which the capsule is implanted.

After sampling and processing, this accelerometric signal will be used, based on the patient's activity, to control the rate of the pacing pulses issued by pacing circuit 38 (controlled pacing of the VVIR type), and/or to carry out a capture test, i.e. to detect the presence or absence of myocardium contraction following the application of a pacing pulse.

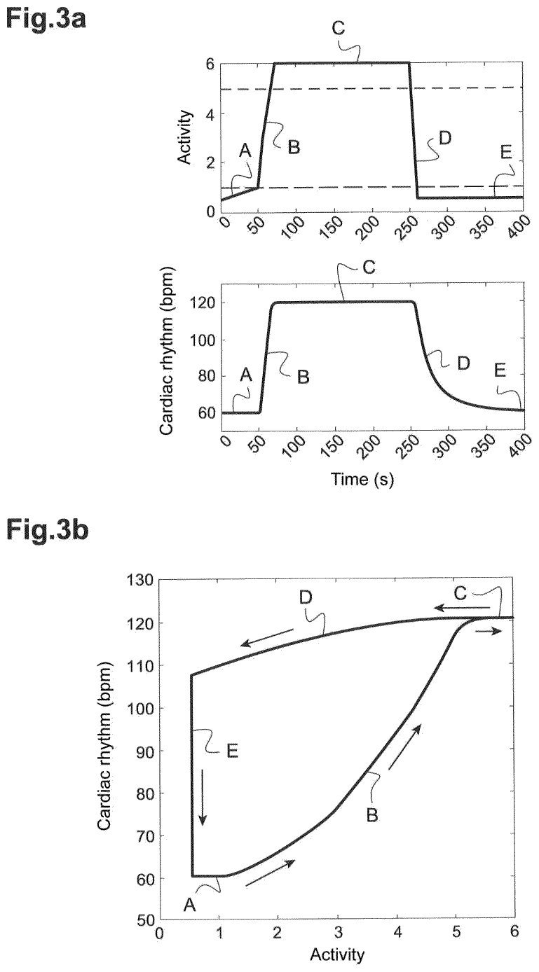

FIGS. 3a and 3b illustrate the way to control as physiologically as possible the patient's activity cardiac rhythm, in a manner comparable to the natural adaptation in a healthy patient.

FIG. 3a is a chronogram showing, in upper part, an example of variation over time of a patient's activity, this activity being represented by an indicator varying from 0 to 5 in arbitrary units. In lower part, FIG. 3a shows the way the heart in a healthy patient adapts to sudden activity variations, in particular the fact that the response to a start of activity is different from the response to a stop of activity. For example, starting from a situation at rest

A at 60 bpm (base rate HRₘᵢₙ at rest), a sudden increase of activity B causes a very rapid increase of the cardiac rhythm, in this example from 60 to 120 bpm (120 bpm being in this example the maximum heart rate HRₘₐₓ of the patient), with a response time of the order of 2 to 10 seconds. During sustained activity, in C, the cardiac rhythm remains at a high level, herein at the maximum rhythm HRₘₐₓ. When the activity decreases suddenly, in D, the cardiac rhythm falls steadily and progressively until it returns to base rate HRₘᵢₙ, in E, with a response time of the order of 3 to 10 minutes. This hysteresis of the response is visible in particular on the activity vs. cardiac rhythm parametric diagram of FIG. 3b, corresponding to the values given as an example in FIG. 3a.

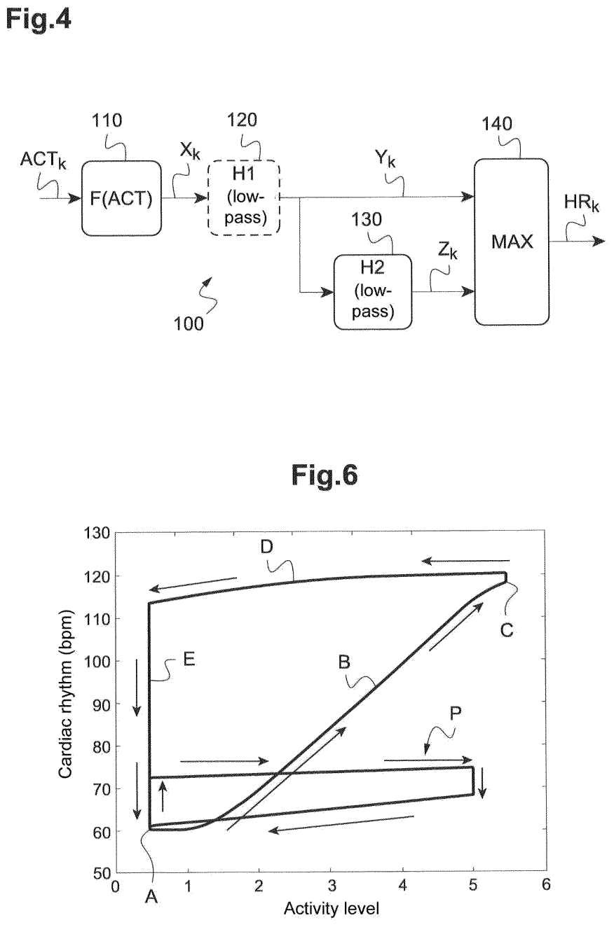

FIG. 4 is a schematic representation, in block form, of the various stages of digital signal processing in a module according to the invention, intended to issue a cardiac rhythm setpoint value to be applied to the input of a control stage of the pacing circuit of an implanted device.

Module 100 according to the invention receives as an input a sampled activity signal ACTₖ. The sampled activity signal is outputted by a detection and digitization module that is not part of the present invention, and that may be of a type known per se, which does not need any particular adaptation for implementing the present invention.

The level of activity of a patient being a parameter that varies over a relatively low frequency range, of the order of 1 to 7 Hz, the sampling rate can be relatively low, of the order of 4 Hz, i.e. 4 samples per second, or less. The activity signal digitization yields a metric that is quantified in a limited number of integer values, for example a metric over 4 or 12 elementary levels.

At time t=k, the sample ACTₖ of the activity signal is applied at the input of a conversion stage 110 that applies to the current value ACTₖ a predetermined activity/cardiac rhythm function F(ACT) and outputs a first target cardiac rhythm value Xₖ.

The target cardiac rhythm having necessarily to be between the minimum value HRₘᵢₙ (base rate) and a maximum value HRₘₐₓ, in the case of a linear activity/cardiac rhythm function, the function F is of the form:

$${X}_{k} = MAX [ {HR}_{min} , MIN ( a . {ACT}_{k} + b . {HR}_{max} ) \} .$$

The values of slope a and y-intercept point b are specific to each patient, and may be parameters that can be configured by the doctor at the time of implantation or during a follow-up visit.

As an alternative, the function F may be a non-linear, monotonic function (as in the example of the arc of curve AC in FIG. 3b corresponding to the natural response of a heart in a healthy patient), or also defined by successive segments, that the doctor can possibly set, for example using a graphical interface on a tablet at the time of implantation or during a follow-up visit.

The cardiac rhythm/activity function F of conversion stage 110 defines a first target cardiac rhythm value Xₖ, but does not define the time interval required to reach this first target value Xₖ. As mentioned hereinabove, to replicate the heart response of a healthy patient, it is desirable:
(i) that the cardiac rhythm increases immediately after the beginning of the activity. A typical example is a seated patient who then stands up and starts to climb a staircase: in such a situation, it is necessary that the cardiac rhythm increases very rapidly. The response time to reach the target cardiac rhythm value will be in this case typically of the order of 2 s to 10 s, depending on the patient's lifestyle (for a patient with an active lifestyle, the response time should be much lower than in the opposite case);
(ii) that the cardiac rhythm decreases slowly and steadily after the activity has been stopped, with a typical time constant of the order of 2 min to 10 min; and
(iii) to ensure immunity to insignificant noises detected by the accelerometer of the activity detection circuit. In particular, signals in a high frequency range, or sudden but transitory changes in the level of activity, must not lead to a rapid change in the cardiac rhythm. For example, in the case of a patient rolling over in bed or coughing, the cardiac rhythm must not be modified, or not significantly modified, even though these episodes will produce a sudden, but brief, variation in the activity detected by the accelerometer.

To meet these various requirements, the target cardiac rhythm value Xₖ will be subjected to a specific filtering processing, detailed hereinafter, provided by stages 120 to 140.

Stage 120 is a low-pass digital filtering stage H1 intended to eliminate the above-mentioned high-frequency noises.

Advantageously, the filter H1 is a recursive exponential filter of the first order, calculating the average of the values Xₖ over a duration τ₁ of the order of 1 or 2 s, according to the sampling rate of the activity detection.

In a simple implementation, and therefore an economical one regarding circuit power consumption, the recursive equation of filtering H1 can be carried out without division nor multiplication, for example by dividing the signal by 8: indeed, such a division corresponds to a simple one-bit shift to the left in the digital value of signal Xₖ.

More precisely, the output of the recursive filter H1 is of the form:

$${Y}_{k} = ( 1 - {α}_{1} ) . {Y}_{k - 1} + {α}_{1} . {X}_{k} ,$$

Yₖ being the current value of Yₖ and Yₖ₋₁ being the previous value in the sequence of samples.

In a division by 8, with a sampling rate of 4 Hz (4 samples per second) and an average calculated over τ₁=2 s, the weighting (1-α₁) will be ⅞ and that of α₁ will be ⅛.

When the next activity sample is acquired, the updating the Y value with the new X value can be done in two operations:

$$\begin{matrix}Y = M * Y - Y + X , and \\ Y = Y / M ,\end{matrix}$$

where M is a rounded integer value of Fₛ×τ₁, Fₛ being the sampling frequency of the detection of activity and τ₁ being the predetermined constant, for example τ₁=1 or 2 s.

If M is taken as a power of 2 (M=2\hat{ }N), the calculation can be performed simply using the following bit manipulation:

$$\begin{matrix}Y = ( Y ≪ N ) - Y + X , and \\ Y = ( Y ≫ N ) ,\end{matrix}$$

where Y<<N represents the binary value of Y shifted by N bits to the left (which is equivalent to multiplying Y by M), and Y>>N represents the binary value of Y shifted by N bits to the right (which is equivalent to dividing Y by M).

The target value Yₖ outputted by stage 12 is applied to a stage 130, that is a low-pass filtering stage H2 intended to take account of the hysteresis of the activity decrease episodes.

Advantageously, filter H2 is a recursive exponential filter of the first order, calculating a moving average of signal Yₖ applied at the input, with a long time constant τ₂, for example 120 s, 300 s or 600 s (value corresponding to the duration of the decreasing time when the activity is stopped).

The output signal Zₖ of filter H2 at stage 130 is given by:

$${Z}_{k} = ( 1 - {α}_{2} ) . {Z}_{k - 1} + {α}_{2} . {Y}_{k} .$$

Z represents the moving average of Y over a duration τ₂, and can be calculated by simple bit shifts as described hereinabove for filtering H1, without costly multiplications or divisions in terms of processor consumption. Once these two filterings H1 and H2 have been applied, two filtered values, respectively Yₖ and Zₖ, are present at the outputs of stages 120 and 130, which values are simultaneously applied at the input of stage 140.

Stage 140 is a combiner stage that outputs the highest of both values Yₖ and Zₖ:

$${HR}_{k} = MAX ( {Y}_{k} , {Z}_{k} ) .$$

HRₖ will be the final target value intended to serve as a setpoint for the cardiac rhythm control by the pacing pulse delivery circuit.

The HRₖ value obtained is a smoothed setpoint value differentiated according to whether we are in a phase of activity increase (in this case HRₖ=Yₖ) or activity decrease (in this case HRₖ=Zₖ).

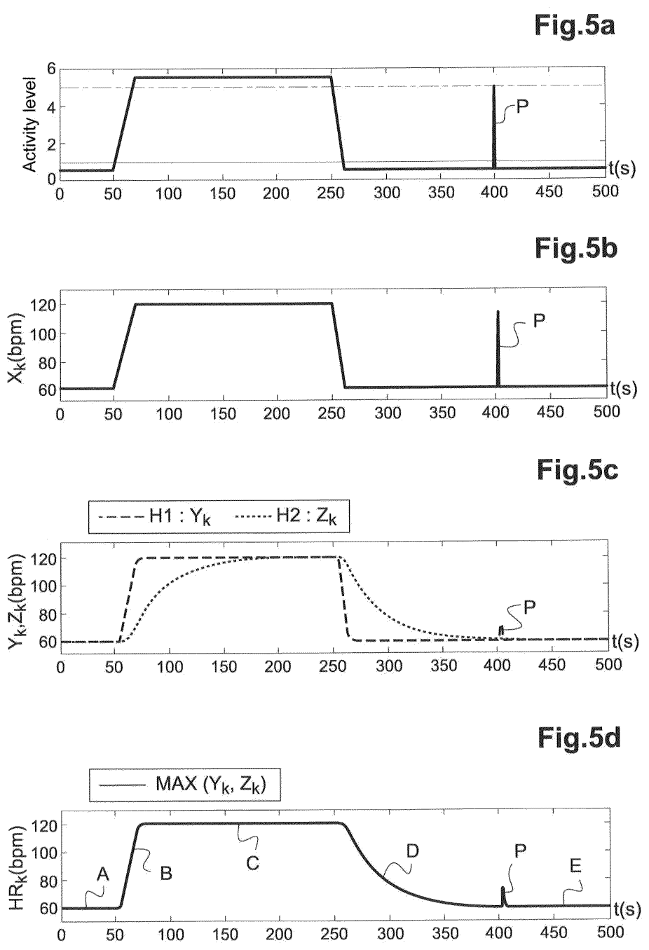

FIGS. 5a to 5d are chronograms illustrating sampled signal values recorded at different levels of the above-mentioned module according to the invention, during detected variations of the patient's activity.

FIG. 5a shows an example of activity variation in a patient, with a sudden increase at t=50 s, the activity stopping at t=250 s. In this figure is also shown, in P, a peak in activity that is high in value but very short (corresponding to a jump, a cough, etc.), thus a parasitic peak for which there is normally no need to modify the cardiac rhythm.

FIG. 5b illustrates the first target cardiac rhythm value Xₖ at the output of stage 110 for activity vs. cardiac rhythm conversion, which therefore tracks variations in activity, including the parasitic peak P.

FIG. 5c shows the corresponding variations of the first and second values of the target signal, i.e. Yₖ at the output of the fist stage 120 of low-pass filtering H1 (dashed line) and Zₖ at the output of the second stage 130 of low-pass filter H2 (dotted line). As can be seen, the filtering H1 has considerably reduced the intensity of the parasitic peak P while preserving the variation in the level of activity.

FIG. 5d shows the final setpoint value HRₖ at the output of the combiner stage 140: in A, in the absence of activity, signal Zₖ is selected; when activity suddenly increases in B, signal Yₖ is selected, and that as long as the activity remains at a high level, in C. When activity suddenly decreases, value Zₖ is selected, with the far longer time constant, in D, thus making it possible to replicate the functioning of the heart in a healthy patient, in a similar way to what was explained and illustrated in FIG. 3a.

FIG. 6 illustrates the variations of the setpoint value HRₖ of FIG. 5d, in cardiac rhythm vs. activity level parametric form.

As can be seen, the peak of activity P produces a significant variation in the curve, but this variation does not lead to a significant change in the cardiac rhythm: in this example, the rhythm increases only from 60 to 70 bpm. On the other hand, when the activity is durably established, in C, at a high level, the cardiac rhythm increases rapidly to its maximum value HRₘₐₓ, here of 120 bpm.

The process described above, which is characteristic of the invention, has a number of advantages.

In particular, the impact of setpoint value calculation on the energy consumption of the circuits is particularly low, in particular if only integer values are used for the calculation (thus without floating point calculation). Moreover, this calculation requires only a very limited number of memory cells, without the need to retain a large amount of history data for the activity signal or the cardiac rhythm signal.

It will finally be noted that, in a simplified version, it is possible to eliminate the first low-pass filtering stage 120, with in this case Yₖ=Xₖ.

This enables the circuits to be further simplified, but with less immunity to parasitic noises of the activity signal collected by the accelerometric sensor. Further, in an alternative embodiment of the invention, the target value calculation is not performed in terms of cardiac rhythm (i.e. rate), but in terms of cardiac interval (i.e. duration), the cardiac interval typically corresponding to the R-R interval between two successive QRS complexes of a heartbeat.

Function F of conversion stage 110 will then be a decreasing function rather than an increasing one (the cardiac interval must decrease when the activity increases), and combiner stage 140 will select the minimum (rather than the maximum) of the filtered cardiac interval values at the output of low-pass stages 120 and 130.

1. A module for calculating a cardiac rhythm, HR, setpoint value, intended to control a rate of pacing pulses issued by an active implantable medical device based on a patient's activity,
wherein the HR setpoint value is continuously determined by digital processing of a sampled activity signal representative of a patient's instantaneous activity,
wherein the module further comprises:
a conversion stage, receiving as an input a current value of the sampled activity signal and outputting a first target HR value, obtained by application of a predetermined activity vs. cardiac rhythm function to a current value of the sampled activity signal;
a first low-pass digital filtering stage, comprising a recursive filter capable of calculating, over a first predetermined duration, a first moving average of the first target HR value issued by the conversion stage, outputting a second target HR value; and
a combiner stage, receiving as an input (i) the first target HR value issued by the conversion stage, and (ii) the second target HR value issued by the first low-pass digital filtering stage, and adapted to determine a maximum of the first and second target HR values received as an input, thereby outputting the HR setpoint value to control the rate of pacing pulses based on the patient's activity.

2. The module of claim 1, wherein the module is integrated to an ASIC circuit.

3. The module of claim 1, wherein the module comprises a microcontroller operating without being put to sleep between two consecutive cardiac cycles.

4. The module of claim 1, wherein the module further comprises:
a second low-pass digital filtering stage, comprising a recursive filter capable of calculating, over a second predetermined duration shorter than the first predetermined duration, a second moving average of the first target HR value, issued by the conversion stage, thereby eliminating parasitic high-frequency noise components that are not representative of the patient's instantaneous activity, and wherein the combiner stage and the first low-pass digital filtering stage receive as an input the first target HR value, after the first target HR value has been filtered by the second low-pass digital filtering stage.

5. The module of claim 1, wherein the predetermined activity vs. cardiac rhythm function is a linear function.

6. The module of claim 1, wherein, to determine the patient's recovery time after a period of intense activity, the first predetermined duration for the first low-pass digital filtering stage to calculate the first moving average is between 120 and 600 seconds.

7. The module of claim 4, wherein, to determine the reactivity of the cardiac rhythm adaptation while eliminating activity noises of very short duration, the second predetermined duration for the second low-pass digital filtering stage to calculate the first moving average is between 1 and 2 seconds.

8. The module of claim 4, wherein the recursive filter of at least one of the first low-pass digital filtering stage and the second low-pass digital filtering stage is an exponential recursive filter of the 1st order.

9. The module of claim 4, wherein the recursive filter of at least one of the first low-pass digital filtering stage and the second low-pass digital filtering stage is a filter capable of calculating a moving average without division nor multiplication.

10. The module of claim 9, wherein the recursive filter of at least one of the first low-pass digital filtering stage and the second low-pass digital filtering stage is a filter capable of operating by shifting bits of the digital representation of the HR value at the input of the filtering stage.

11. The module of claim 1, wherein the module is integrated to an active medical device of the implantable autonomous capsule type which houses, in a device body, an electronic unit including:
a pacing circuit, capable of issuing pacing pulses;
a circuit capable of issuing a signal representative of the instantaneous activity of a patient wearing the device;
a module for sampling or extracting a sampled activity signal from the signal representative of the patient's instantaneous activity; and
said module, to control a rate of the pacing pulses issued by the pacing circuit based on the patient's activity, by calculation of a HR setpoint value.

12. A module for calculating a cardiac interval, RR, setpoint value, intended to control a rate of pacing pulses issued by an active implantable medical device based on a patient's activity,
wherein the RR setpoint value is continuously determined by digital processing of a sampled activity signal representative of the patient's instantaneous activity,
wherein the module further comprises:
a conversion stage, receiving as an input a current value of the sampled activity signal and outputting a first target RR value, obtained by application of a predetermined activity vs. cardiac interval function to a current value of the sampled activity signal;
a first low-pass digital filtering stage, comprising a recursive filter capable of calculating, over a first predetermined duration, a first moving average of the first target RR value issued by the conversion stage, outputting a second target RR value; and
a combiner stage, receiving as an input (i) the first target RR value issued by the conversion stage, and (ii) the second target RR value issued by the first low-pass digital filtering stage, and adapted to determine a minimum of the first and second target RR values received as an input, thereby outputting the RR setpoint value, to control the rate of pacing pulses based on the patient's activity.

13. The module of claim 12, wherein the module is integrated to an ASIC circuit.

14. The module of claim 12, wherein the module comprises a microcontroller operating without being put to sleep between two consecutive cardiac cycles.

15. The module of claim 12, wherein the module further comprises:
a second low-pass digital filtering stage, comprising a recursive filter capable of calculating, over a second predetermined duration shorter than the first predetermined duration, a second moving average of the first target RR value, issued by the conversion stage,
thereby eliminating parasitic high-frequency noise components that are not representative of the patient's instantaneous activity,
and wherein the combiner stage and the first low-pass digital filtering stage receive as an input the first target RR value, after the first target RR value has been filtered by the second low-pass digital filtering stage.

16. The module of claim 12, wherein the predetermined activity vs. cardiac interval function is a linear function.

17. The module of claim 12, wherein, to determine the patient's recovery time after a period of intense activity, the first predetermined duration for the first low-pass digital filtering stage to calculate the first moving average is between 120 and 600 seconds.

18. The module of claim 15, wherein, to determine the reactivity of the cardiac rhythm adaptation while eliminating activity noises of very short duration, the second predetermined duration for the second low-pass digital filtering stage to calculate the first moving average is between 1 and 2 seconds.

19. The module of claim 15, wherein the recursive filter of at least one of the first low-pass digital filtering stage and the second low-pass digital filtering stage is an exponential recursive filter of the 1st order.

20. The module of claim 15, wherein the recursive filter of at least one of the first low-pass digital filtering stage and the second low-pass digital filtering stage is a filter capable of calculating a moving average without division nor multiplication.

21. The module of claim 20, wherein the recursive filter of at least one of the first low-pass digital filtering stage and the second low-pass digital filtering stage is a filter capable of operating by shifting bits of the digital representation of the RR value at the input of the filtering stage.

22. The module of claim 12, wherein the module is integrated to an active medical device of the implantable autonomous capsule type which houses, in a device body, an electronic unit including:
a pacing circuit, capable of issuing pacing pulses;
a circuit capable of issuing a signal representative of the instantaneous activity of a patient wearing the device;
a module for sampling or extracting a sampled activity signal from the signal representative of the patient's instantaneous activity; and
said module, to control a rate of the pacing pulses issued by the pacing circuit based on the patient's activity, by calculation of a RR setpoint value.
